(19) United States
(12) Patent Application Publication
(10) Pub. No.: US 2023/0323441 A1
(43) Pub. Date: Oct. 12, 2023

(54) Rapid Simplified PCR Point-of-Care Cartridge System with Highly Sensitive Fluorescent Reading Optics Which Starts Reading at the First Thermal Cycle.

(71) Applicants: William D. Bickmore, St. George, UT (US); Neil D. Holland, St George, UT (US)

(72) Inventors: William D. Bickmore, St. George, UT (US); Neil D. Holland, St George, UT (US)

(21) Appl. No.: 17/715,705

(22) Filed: Apr. 7, 2022

(51) Int. Cl. C12Q 1/686; B01L 7/00; B01L 3/00

(52) U.S. Cl. CPC C12Q 1/686; B01L 7/52; B01L 2400/0481; B01L 2300/1827; B01L 2400/0478; B01L 3/50273

(57) ABSTRACT

A single-use special-purpose disposable vertically operated Polymerase Chain Reaction (PCR) cartridge that automatically performs extraction of Deoxyribonucleic Acid (DNA) or Ribonucleic Acid (RNA) using focused ultrasound and its associated transient cavitation pressure followed by automatic purification and distribution to a plurality of PCR thermocycling vials. The cartridge utilizes a novel optical system that allows reading of the PCR binary expansion of Double-Stranded DNA (dsDNA) at early cycles usually considered unreadable background noises resulting in not only exceptionally fast sample preparation but very quick data confirming positive or negative samples in as little as 10 cycles. The PCR vials contain lyophilized, pellets comprising all the necessary chemicals for PCR. This process requires no ancillary equipment such as microfuges, or pipettes. Once the DNA or RNA containing sample and the processing fluid are added to the cartridge, all else is automatic. The process fluid passes through each part of the cartridge only once. There are no revolving mechanical parts or otherwise complex mechanical systems. The cartridge does not vent into the atmosphere and minimizes environmental contamination. The cartridge is tightly sealed to prevent leakage.

### BASIC OPERATION OF THE CARTRIDGE

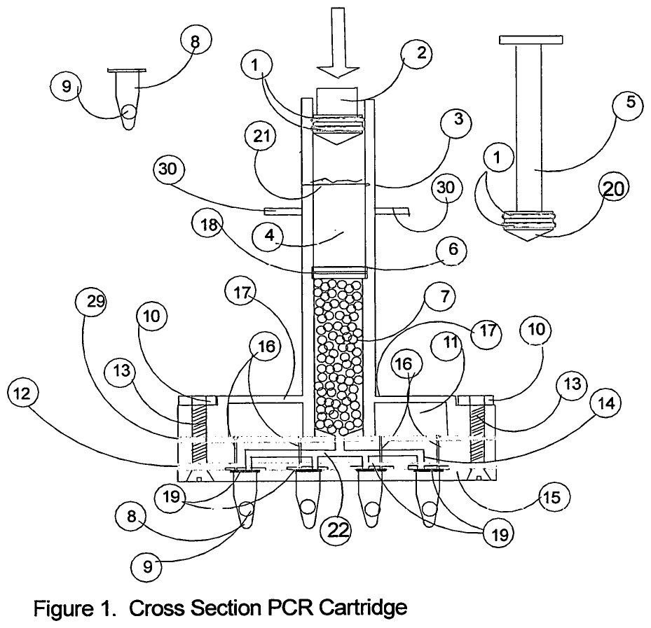

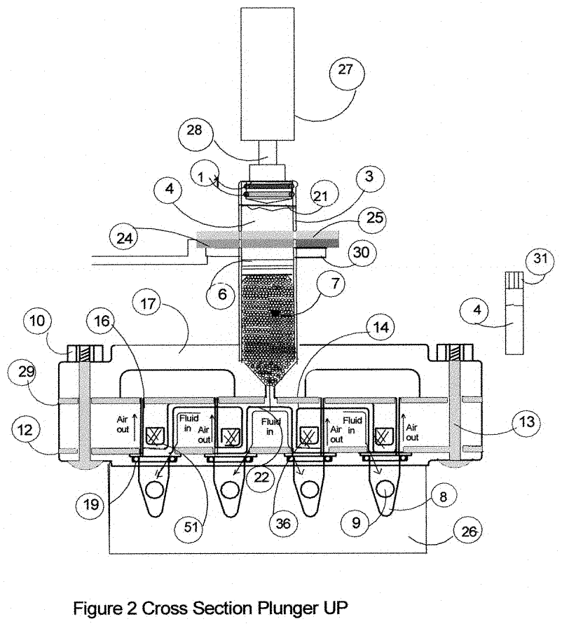

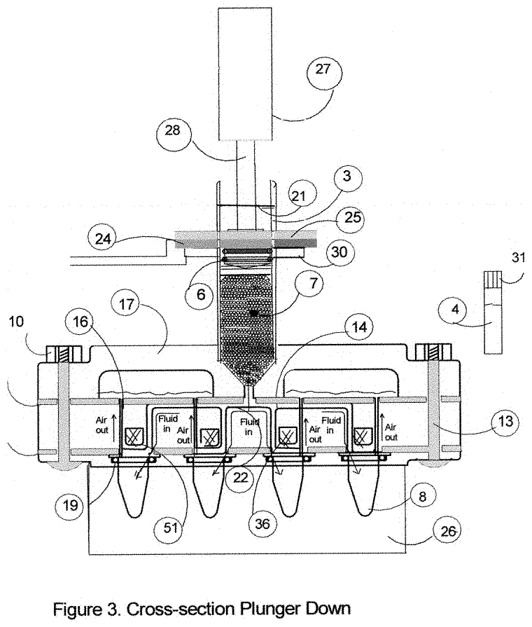

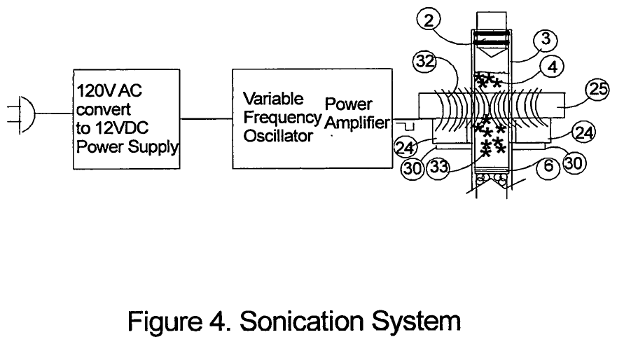

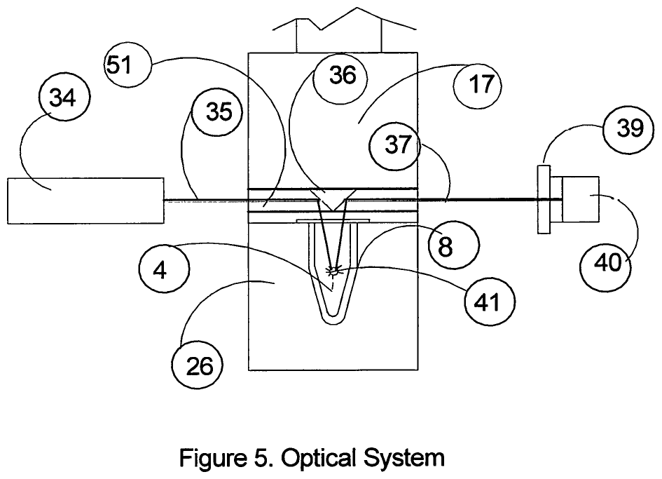

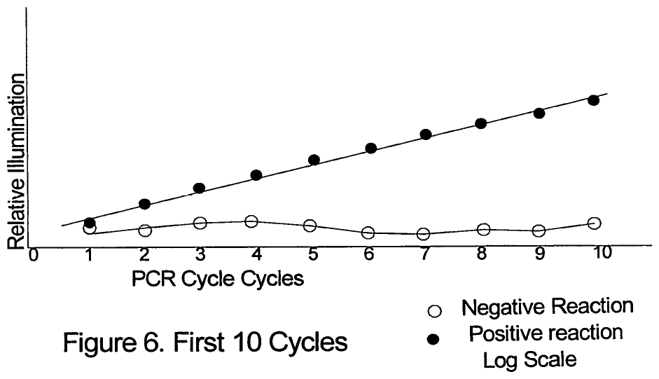

PCR is a complex chemical reaction. Conventionally extraction and purification of the DNA or RNA sample must be performed in a laboratory before thermocycling as independent time-consuming steps. This invention, as illustrated in FIG. 1 performs these steps automatically and fills a plurality of PCR vials (8) ready for thermocycling. Since the cartridge (17) is vertical and filled from the top, the surplus of air above the fluid level is not forced through the purification column into the lower parts of the cartridge (17) and therefore does not add to the burden of trapped air to the lower parts of the cartridge (17). The DNA or RNA to be amplified, while yet in their native cellular or viral form, is directly added to the barrel (3) after fluid (4) is poured into the barrel from a 2 ml centrifuge tube (31). Piston (2) or Piston (5) is then inserted into the barrel (3) and seals the cartridge (17). Once sealed the cartridge (17) prevents the escape of pathogens or amplicons. The cartridge (17) may use only a single PCR vial (8) or a plurality of vials. The preferred embodiment comprises 4 PCR reaction vials. When used with a general-purpose thermocycler, the thumb activated piston (5) which is attached to another preferred embodiment of the piston can be used much like a syringe to apply the necessary pressure to drive the fluid through the purification column (7). The cartridge (17) becomes fully automatic when operated with a thermocycler designed expressly for this cartridge (17). It provides the mechanical driving force through an electronic linear actuator (27) for the cartridge (17) to operate (FIGS. 2 and 3). FIG. 4 shows an ultrasonic transducer (24) and its associated electronic driver provide the necessary transient cavitation pressure to extract the DNA or RNA from its source. After the cartridge (17) is fully prepared and inserted into a thermocycler, the ultrasonic transducer (24) and its attached horn (25) are brought down over the barrel (3) and the horn is snuggly in contact with the barrel. Its final operating position is directly above the cartridge tabs (30). The fluid (4) level must be higher than the horn (25) to allow the ultrasonic energy to be transmitted to fluid (4). Once thermocycling begins, at the end of the first cycle, the first fluorescent reading will be taken. The highly sensitive optical system (FIG. 5) can read optical fluorescent signals indicating the presence of dsDNA. Sybr®Green or similar indicators and/or target specific DNA fluorophores which have become active after their quenching components have been removed during thermocycling are used to illuminate the target dsDNA. Most optical systems do not have the ability to read the small optical signals from the first cycles of PCR. This is commonly known by those familiar with the art as background noise. Once the signal becomes strong enough to cross over a predesignated crossing threshold, this is commonly known as coming out of the background. This invention allows not only for the rapid preparation of specimens for PCR but allows sufficient fluorescent optical signal to be read and interpreted as indicative of a positive-going sample from one which does not grow and increase with successive cycles. FIG. 6 represents cumulative trials of data taken from a log-based optical sensor of the first 10 cycles of a thermocycler. The upward-sloping successive data points correlate with conventional fluorescent thermocycler readings taken from later cycles which have clearly crossed a conventional processing threshold and indicate a positive PCR test. Likewise, the data which does not increase in signal during the first 10 cycles correlates closely with conventional fluorescent thermocycler readings or a negative test. The intent of this invention is to provide a quicker point-of-care PCR test which is either positive or negative. It is designed to be used where conventional PCR testing may require more time than is expedient for determining an outcome of the test. This enables the proper treatment to the patient to begin much more quickly than is possible with conventional PCR which may require several hours to days before the test data becomes known.

### Technical Overview

This cartridge (17) may be used by technicians with limited PCR experience. It is designed to significantly de-skill the PCR process. The cartridge's simplified vertical design has no moving parts except for a simple plunger which operates much like a syringe. This cartridge (17) requires no ancillary equipment such as a microcentrifuge or pipetting equipment. Extraction of DNA or RNA is accomplished ultrasonically. The ultrasonic energy is produced by a novel flat disk ultrasonic horn and a flat ring-shaped transducer (24) such as American Piezo 70-222 bonded to the ultrasonic horn. This design incorporates Bickmore et al U.S. Pat. No. 8,169,122 assigned to Bickmore. It uses the principles of ultrasonic induced transient cavitation pressure wherein DNA or RNA are quickly released from microbial, viral, or cellular sources without the use of conventional chemistry for extraction. It does not require an ultrasonic probe to be introduced into the cartridge (17).

Traditional extraction chemicals may be carcinogenic. Since no traditional extraction chemicals are used in the process, it does not require the removal or neutralization of extraction chemicals that could otherwise interfere with the PCR process. It does not require the elusion of DNA or RNA from glass fiber or other substrates commonly practiced in PCR preparation protocols.

The cartridge (17) utilizes lyophilized self-contained chemistries (9) which are shelf-stable without refrigeration such as those produced by Bio-lyph of Chaska, Minnesota. It is designed to be used whenever rapid or simplified PCR testing is desirable. It is particularly useful in point-of-care PCR systems or portable PCR systems and may be used in a wide variety of environments and remote applications. When coupled with a companion high-sensitivity fluorescence reader the overall speed of the test is greatly reduced compared with conventional PCR.

The cartridge (17) does not vent into the atmosphere. This greatly reduces the possibility of pathogens and/or amplicon contamination of the laboratory or field location and reduces the danger of conveying pathogens or amplicons to the laboratory or the laboratory technician. Chambers (11) capture expelled gasses and small amounts of excess liquid. The vents (16) which lead from the reaction vials (8) to the chambers (11) have small-bore narrow passages (16) which allow air to freely pass out of the vials (8) into the chambers (11) but require additional force for fluids to pass through the restricted bore. This mechanism ensures that all the PCR vials will be filled with fluid (4), replacing the entrapped air before tiny amounts of excess fluid (4) are allowed to escape into the chambers (11). With the air properly vented under pressure, the aqueous fluid (4) passes through the purification median (7) and then evenly enters the reaction vials (8) and hydrates the lyophilized PCR chemistry (9). The lyophilized chemistry (9) is processed under anhydrous nitrogen. The cartridge (17) is packaged in a heavy foil and polymer bag to exclude all moisture until it is rehydrated with fluid (4).

The optical system of FIG. 5 allows for the rapid reading of the first 10 cycles of PCR in what is ordinarily considered background and unreadable. This enables a much shorter test for point-of-care where the basic positive or negative information is all that is required. There exists a possible way of determining quantity of DNA in the first 10 samples, but this will be explored at a later time.

### A BRIEF DESCRIPTION OF THE PARTS OF THE PCR CARTRIDGE

Refer to FIGS. 1, 2, 3, 4, 5, and 6
1. Piston O-rings.
2. The Piston and its associated O-rings act very much like a syringe. Piston (5) is either manually pushed or a piston (2) is pushed using the actuator (27) to force fluids through the cartridge (17) purification median (7) into the distribution channels (22) and fill the reaction vials (8) completely with fluid (4) and dissolves lyophilized PCR chemistry (9).
3. The Barrel (3) with a slight external taper from top to bottom to accommodate the uses of an ultra-sonic transducer and horn more fully described hereinafter. The barrel also has a slight internal prominence at a mid-point which is a support ring to provide a stop ring to position filter housing (6). The filter housing (6) provides a mounting of a sub-micron membrane filter (18). The purpose of the sub-micron filter (18) will be described hereinafter.
4. PCR aqueous buffer fluid (4) receives added biological source of dsDNA or RNA while the barrel is yet open. Fluid (4) may contain salts or minerals necessary for PCR such as magnesium chloride.
5. When it is desirable to operate the cartridge (17) manually, the thumb-powered piston (5) can be used in place of an electronic linear actuator to apply pressure in a downward direction indicated by the arrow in FIG. 1.
6. The two-part filter support mechanism (6) supports the sub-micron hydrophobic filter (18). The purpose of the filter (18) is to provide a barrier against fluid (4) entering the next stage of the cartridge (17) prior to fluid (4) being sonicated to release dsDNA and or RNA. After sonication has taken place, sufficient pressure will be placed on the piston to drive the fluid through the hydrophobic filter (18). Pressure will be applied to the piston at approximately 20 psi which will cause the filter (18) to hydrate and pass fluid (4) into and through the purification column (7) medium.
7. A purification column (7) such as manufactured by Zymo, allows DNA to freely pass through the purification column while retaining proteins, PCR inhibitors and other impurities. In the case that RNA is produced during sonic extraction, it is purified and enzymatically converted to dsDNA via reverse transcriptase. The appropriate column material must be provided that is suitable for the processes required.
8. A single or a plurality of PCR reaction vials (8) with a flange and gasket (19) at the top, along with gasket (12), prevent the escape of fluids. These vials are spaced and of a size to be compatible with the size and spacing of commercially available strips of PCR vials. The cartridge (17) may be assembled with either a single vial or a plurality of vials attached to the cartridge (17) as shown in FIG. 1. depending upon the number of simultaneous PCR tests to be run through the cartridge (17). When the cartridge (17) is used with highly sensitive optical systems more particularly described hereinafter, they must be made of polymers that have very little native fluorescence. Typically, they are black in color.
9. Lyophilized chemistry pellet (9) either singularly within one vial (8) or a plurality thereof, are previously prepared lyophilized and maintained and handled in an anhydrous nitrogen low moisture atmosphere prior to being placed in foil and polymer pouches which positively prevent moisture incursions. Pellets (9) also contain lyophilized up-hill and downhill primers and fluorescent probes and their associated quenchers specific to the tests to be run and all other components of the PCR chemistry not contained in the fluid (4).
10. Machine nuts (10) together with machine screws (13) secure the cartridge (17) and its various components. To remain properly assembled they allow the cartridge gaskets (29)(12), the manifold (14), and cartridge bottom plate (15) to be secured, when tightened. Other forms of mechanical attachment may be used such as the following, but not be limited to; rivets, snap-together ridged thermoplastic components, adhesive bonding, or thermal bonding afforded by laser or ultrasonic bonding and similar or any means of reliably bonding components.
11. Closed system purged air collection chambers (11) allow air to accumulate and be compressed when fluid (4) is expressed into the PCR reaction vials (8). The captive air naturally rises into the chambers (11) through capillary tubes (16) as the fluid is introduced under pressure.
12. A lower gasket (12) constrains fluid (4) to pass through designated fill channels (22).
13. Machine screws (13) together with machine nuts (10) allow the cartridge (17) components to remain securely assembled.
14. Manifold (14) fill channels (22) distribute fluid (4) into a single or a plurality of PCR vials (8).
15. A cartridge (17) bottom plate (15) is used to secure parts of the cartridge (17) into a functional device.
16. PCR reaction vial capillary air exhaust ports (16) which are of small enough diameter to act as capillary tubes that freely pass air but restrict fluids, provide a small differential back pressure of about 0.5 psi to allow the air to be first exhausted into the purged air collection chambers (11) and thereafter when all vials (8) are successfully filled with liquid (4) and under sufficient pressure to then pass a small amount of overflow of liquid (4) to purged air retention chambers (11). The internal pressure in the purged air retention chambers will be between 25 PSI and 60 psi.
17. Assembled cartridge (17) body.
18. Sub-micron hydrophobic membrane filter (18) as described in the two-part filter support mechanism (6).
19. Gasket (19) atop the flange of a single or a plurality of PCR reaction vials (8).
20. The conical piston bottom is an ultrasonic reflection element on the face of the pistons (2) and (5).
21. Maximum fluid fill line (21) marked on the side of the cartridge (17) barrel (3).
22. Fill channels (22) pass fluid (4) through the manifold (14) to the vials (8).
23. Not used.
24. While not being bound by theory, the ultrasonic flat ring-shaped transducer (24) is a piezoelectric device that transforms the electronic energy at approximately 40 kHz to mechanically propagate ultrasound waves. Electrical leads attach the transducer to the output of the driving amplifier. At full capacity, it operates at about 35 Watts. It is driven by a frequency generator that is tunable to the resonant frequencies of the transducer. Sonication may last from a few seconds to a few minutes and is used to release DNA and/or RNA from microbial sources or cellular sources. It is suspended on standoffs (30) which are attached to the barrel (3). The transducer is epoxy bonded to a flat disk aluminum Horn (25).
25. The horn (25) comprises a circular flat metal disk with a central lumen which is a force-fit to the outer circumference of the barrel (3). It transmits and focuses the ultrasonic horizontal energy from the ultrasonic transducer (24) to the barrel sidewall which in turn radiates the ultrasonic energy into the fluid (4). The ultrasonic energy creates cavitation bubbles (33) in the fluid (4) which arise and then spontaneously collapse under the action of the ultrasonic sound waves. The ultrasonic sound wave creates high and low-pressure points in the fluid as the sound wave passes through. The cavitation bubbles are formed by the low-pressure part of the soundwave and expire upon the next high-pressure part of the sound wave. When a cavitation bubble collapses, it releases a high-pressure microjet of fluid and a photon. This jet of fluid hydraulically abrades the surfaces of the cellular structures within the fluid or the external parts of a virus or bacteria and releases genetic material into the fluid (4). The size of the cavitation bubbles is dependent on the frequency of the ultrasound waves. Lower frequencies create the largest cavitation bubbles. If higher frequencies in the megahertz area are used, cavitation is practically extinct.
26. The Heating and cooling metal block (26) is a standard component of a thermocycler which is designed to alternately heat and cool the fluids (4) in the reaction vials (8) in repeated cycles designed to produce DNA amplification of a targeted segment of DNA when it is present.
27. An electronic linear actuator (27) has a motor-driven central extension rod (28) which is a mechanically downward-driven piston (2) providing the mechanical power to propel fluid (4) movement through the cartridge (17). Alternately, a piston (2), with a manual push rod (5) may be used to drive the fluid movement through the cartridge manually when a linear actuator is not present.
28. The extension rod (28) transmits the mechanical force of the actuator (27) to the piston (2) which in turn will move the fluid (4) through the sub-micron hydrophobic membrane filter, the purification column (7) and then into the reaction vials (8).
29. Upper manifold gasket.
30. Standoff tabs (30) on the barrel allow a place for two fingers to assist the manual operation of the cartridge (17) with the thumb pressing on the manual piston (5). These same standoff tabs (30) establish a limit to the bottom position of the ultrasonic transducer (24).
31. The sample fluid (4) contents of the 2 ml capped centrifuge tube (31) is poured completely into the barrel (3) of the cartridge (17) then the biological samples are added to the existing fluid (4) in the barrel (3).

The following numbers are illustrated in FIG. 4.
32. Represents sound waves (32) traveling through the horn (25) which firmly surrounds barrel (3) holding liquid (4).
33. Represents cavitation bubbles (33) forming in fluid (4) during sonication.

The following numbers are illustrated in FIG. 5.
34. A low-wattage compact laser (34) or high-powered LEDs with required excitations of specific florescent frequencies of a wavelength that excites the fluorophore that is used to identify either dsDNA or a particular segment of dsDNA which is a PCR target to replicate when present.
35. The excitation light (35) emitted by the laser or LED (34) passes through a clear light channel (51) embedded in the cartridge (17), is deflected by a mirror assembly (36) into each vial (8) wherein the light is designed to excite the fluorescent materials (41) in the reaction vials (8). The emission light (37) then passes out of the vial (8) and is deflected by the mirror assemble (36) through a continuance of the clear light channel (51) out to the high-band pass filter (39) to the high sensitivity solid-state low-light-sensitive photon receiver (40).
36.-40 & 51. (The clear light channel (51) is discussed in 35 above.) Mirror assembly (36) comprising two primary surface mirrors on a wedge-shaped backing; one mirror to reflect the excitation wavelength of light (35) produced by the laser or LED (34) down to the fluorescent materials (41) suspended in the vial (8) fluid (4), and a second primary surface mirror to reflect the fluorescence emission light (37) coming from the fluorescent materials (41) is shifted according to the physics of fluorescent materials 20 nanometers or more higher than the excitation wavelength. The emitted light wavelength (37) is reflected by a second primary surface mirror on mirror assembly (36) from the fluorophore (41) such that it passes through the high-band pass filter (39) which blocks the excitation wavelengths (35) while admitting the emitted wavelengths of light (37). Said emitted light travels through the filter (39) and strikes the photosensitive surface of the photo receiving element (40). The excitation beam (35) is reflected at an angle of 95-97° at the first primary surface mirror (36) into the vial (8). The emission wavelength beam (37) from the fluorophore (41) is reflected at 95-97° angle from the emission of the fluorescent materials (41) by the means of the second primary surface mirror (36) through the high-band pass filter (39) to the high sensitivity solid-state low-light-sensitive photon receiver (40). The detected emitted light intensity is measured at the end of each thermal cycle. Light reading increases are detected beginning at the second thermal cycle and continue to indicate a consistent increase as more dsDNA is created through the processes of PCR. The low-light limit of detection is capable of showing increases in emitted light throughout what has previously been termed “background” emissions not detected by most Real-time (qPCR) fluorescent-reading thermal cyclers for several cycles until their limit of detection has been reached.

The results of this accelerated PCR test are available to the clinician much sooner than by traditional means. Traditional PCR machines may take 2 or more hours to determine the outcome of the tests. Moreover, most PCR equipment today is far away from the point where the sample was taken. Shipping and processing of the sample may take days to weeks. These conventions make it impossible to give prompt information which might enable a person who is infected with the disease but is yet to be symptomatic to make knowledgeable decisions to avoid infecting others. An asymptomatic person may unwittingly spread disease while not yet manifesting symptoms. Prompt PCR has the capacity to reduce the spread of disease from asymptomatic persons who are nonetheless infected by providing necessary information. Conventional PCR tests provide delayed response to infected persons in the term of days and weeks. This is added to the laborious conventional preparation steps which may take more than an hour. By contrast, the combined efficiencies of this invention result in positive or negative PCR tests in terms of minutes. PCR testing typically occurring during the Covid-19 SARS virus pandemic took days to weeks to confirm the presence of the SARS virus. This allowed the infected person to continue to expose others to the disease before the presence of the virus could be confirmed. This very problem allows the virus to be spread until a detectable level of antibodies is reached by the infected person to establish that a presumptive case of viral infection has been demonstrated via antibody test. Moreover, after many days pass, the presence of the virus may′finally be confirmed by PCR.

This invention promptly confirms whether a virus is present much faster than the present systems for PCR in practice. This invention when combined with a compatible PCR thermocycler can provide rapid clinical information that can curtail the spread of the disease during the virus infection latency period. This is a period during which the antibody titer is low and not yet detectable, yet the virus is reproducing at very rapid rates. The patient may not yet feel sick but can spread the disease to others before knowing the patient is actually producing vast numbers of viruses. The infection may be well established before the patient is symptomatic. The antibody tests are not reliable early-stage indicators. Antibody levels are also limited in determining if the virus has been eliminated from the host. The lack of PCR machines at the point-of-care which can give definitive answers in minutes while the patient waits for the result, allows for the inadvertent spread of high morbidity and/or a high mortality infection and may result in a pandemic.

The Cartridge Kit:
1. The dry portion of the cartridge kit (17) is prepared in advance with all components assembled and ready to operate. The plurality of lyophilized PCR chemistry (9) pellets are also prepared in advance then placed in the reaction Vials (8). Great care must be taken not to expose any lyophilized materials to the slightest measurable humidity before using the cartridge. Lyophilized materials should only be handled in an anhydrous nitrogen atmosphere when not in a sealed cartridge. The dry portion of the cartridge is then placed into a foil and polymer pouch and hermetically sealed.
2. The liquid (4) comprising PCR grade water and mineral salts, such as magnesium chloride are placed in a sealed cap centrifuge tube (31) and then into a foil and polymer pouch and then hermetically sealed. This comprises the liquid portion of the test kit.
3. Both the dry and liquid sub-assemblies of the test kit are placed into a container in equal numbers.
4. The container is then properly labeled, closed, and sealed then stored at room temperature until used.

The PCR Process with the Single-Use Cartridge (17):
1. To run a test with the cartridge, the PCR technician must remove from the kit container one of each of the subassemblies of a test.
2. Open and remove each of the two sub-assemblies from its respective sealed foil pouches with sterile gloved hands on a clean surface.
3. Remove the piston (2) from the barrel (3) and pour the liquid (4) contents completely out of the capped 2 ml centrifuge tube (31) into the barrel (3) of the cartridge (17). The test sample is then introduced into the liquid (4) within the barrel (3). If a wooden-handled specimen collection swab is used to collect mucus and cells or pathogens, after swabbing the desired area twirl the stem of the swab several times between the thumb and index finger of a gloved hand while the tuft of the swab that is submerged in the fluid (4). Properly prepared samples of a variety of biological forms may be judiciously introduced into the liquid using non-contaminating techniques.
4. After introducing the fluid (4) and the test sample, into the barrel, replace the piston with either the embodiment of a piston designed to be used with manual actuation (5) or the embodiment of the piston (2) to be automatically driven (28) with a linear actuator (27).
5. Place the cartridge (17) with its PCR reaction vials (8) in the thermocycler block (26) and secure it.
6. The ultrasonic transducer (24) and the ultrasonic horn assembly (25) are placed on the barrel (3) through the lumen of the transducer (24) and the lumen of the horn (25) by pushing down the transducer (24) and the horn (25) assembly until the horn (25) fits snugly on the barrel (3).
7. If an electronic linear actuator (27) and push rod (28) will be used, affirm they are aligned to activate the piston (2).
8. Once all system components are in the starting position to process the sample, set the timer function of the sonicator for a prescribed time ranging from a few seconds to several minutes as specified for the test kit being used.
9. Sonication is initiated. Sonication comprises the generation of short-lived high-energy cavitation bubbles which, upon collapsing, release strong cavitation origination micro jet forces to abrade cellular structures, bacterial cell walls, or virus coverings with sufficient force to hydraulically abrade these structures and release the DNA or RNA.
10. Upon completion of the sonication step, the actuator (27) or manual thumb piston (5) will move fluid (4) by first generating sufficient pressure to wet the hydrophobic submicron membrane (18) and subsequently slowly push the fluid (4) through the purification column (7) into the distribution manifold (14).
11. The piston (2) will continue to slowly descend until it reaches its limit.
12. After the fluid passes through the purification column, it will then pass out of the barrel into the distribution manifold and then fill the PCR reaction vials (8) hydrating the PCR chemistry pellets (9).
13. As the PCR vials (8) fill through the channels (22) of the manifold (14) captive air is expelled from the PCR vials to the air retention chambers (11). The air must pass through narrow openings (16). These small capillary-like openings are designed to create a differential pressure that restricts the flow of liquid more than the flow of air. Once the vials (8) are filled, a small amount of the fluid will be expressed through the capillary tubes (16) under slightly increased pressure as the piston (2) completes its prescribed travel.
14. With the cartridge now prepared and ready. PCR thermocycling may begin.
15. The ideal yield of duplicated targets formed by PCR with thermocycling follows the simple mathematical expression of 2n which is defined as 2 raised to the power of n, where n is equal to the number of thermal cycles performed by the thermocycler. The number of thermal cycles can be significantly reduced with this invention that gathers meaningful low-light signal information from within what was previously considered unreadable “background noise”.

The Optical System:

See FIGS. 5 and 6

The cartridge (17) has a novel optical system that allows reading of the fluorophores beginning at the end of cycle 1 and continuing for up to 10 cycles or more if desired.

Historically, the useful fluorescent light signal coming from the earliest cycle counts of PCR has been referred to as “background” emissions and occurs on such a small scale that they have been generally ignored as not useful in data collection. However, the fluorescent light signal increases generally with the increase in DNA active fluorescent indicators mathematically following the formula of 2ⁿ where n is the cycle count. When the efficiency of PCR is less than 100 percent the signal will increase somewhat slower than 2ⁿ. However, the inventors using this special optical system have measured discernable transitions from as low as the first cycle to the second cycle of duplicated DNA and on up to the tenth cycle. Generally, the “background signal” is presumed to extend to the 11^{th} cycle or greater. At some point in the cycle count, an operator or artificial intelligence will declare a “crossing threshold”. This is normally the point where the photons emitted by the fluorescent dyes attached to the DNA are historically considered to be readable and first confirms that the desired section of DNA called the “Target” is present in the sample and being duplicated by PCR when the fluorescent indicator signal is strong enough to exceed the “crossing threshold”. The number of the cycle at which the crossing threshold is first crossed may be used to quantify the amount of target DNA present in the initial sample. That is, the amount of target DNA being replicated is mathematically imputed to be greater if the crossing threshold is crossed at an earlier cycle such as cycle 15 or 16 than when the cycle count is greater such as cycle 20 or 21 before the threshold is crossed. The original load of the target segment is computed to be smaller when the fluorescence cycle number is greater before the fluorescent signal crosses the threshold because it has taken more cycles of duplication of the target to achieve a signal level strong enough to cross the designated crossing threshold.

Correlation of Low-count Cycle Data:

See FIG. 6

The slope of the data taken from the first 10 cycles has demonstrated a high degree of correlation with the conventual methods used by conventional qPCR machines to determine whether the test is positive or negative for the target DNA. However, the optical system must be orders of magnitude more sensitive than conventional qPCR machines. This necessitates using cartridges or test wells (8) that have very low native fluorescence and are typically pigmented with black dye to increase the signal-to-noise ratio. It is also important to increase the number of excitation photons impinging upon the fluorophore (41), such as a miniature laser or LED (34) which radiates at the proper wavelength. On the reading side of the fluorophore, the emission photons must be properly directed to a high-band pass filter (39) which blocks the excitation wavelengths (35) and passes the shorter wavelengths (37) which are emitted by the fluorophore (41). Two primary-surface mirrors on the mirror assembly (36) direct the excitation light beam (35) to the fluorophore (41) at an angle of 95-97° and redirect the emission photons (37) of the fluorophore (41) at an angle between 95-97° towards the high-band pass filter (39) and the highly sensitive photon light reader (40) which may be a solid-state photomultiplier device, an avalanche diode, or similar low light detectors. Primary surface mirrors (36) are used to reduce signal loss and improve accuracy.

Time is of The Essence:

In recent times, the world has been thrust into a pandemic of unprecedented infection rates. PCR typically consists of thermocycling the test materials in the presence of the proper mix of chemicals and oligos to allow the replication of a certain segment of DNA if it is present. Viral genetics may be in the form of RNA which must be converted to DNA prior to target amplification. A typical qPCR device will require about 1 to 2 minutes of time for each cycle or approximately 40 to 90 minutes after the prep step is completed. The prep step may add as much as an hour to the process. The use of the cartridge described in this patent may shorten the prep-time to around 1-2 minutes. Using the optical system of the cartridge described herein can shorten the thermal cycling to around 10 minutes and take adequate and meaningful data which would correlate closely with qPCR machines which may take upwards of two hours to yield the equivalent test results. Many days are often used to ship PCR materials to a regional test site resulting in contagion being unwittingly spread by a person who has contracted the disease but has yet to express symptoms. When ultimately confirmed by PCR tests, taking up to two weeks to read may lead to many more people being contaminated than would be the case if a single-use cartridge was used at the point of care and would reveal the answer in 10 minutes or less. If a plurality of smaller PCR portable thermocyclers were widely distributed utilizing the cartridge described in this patent pandemics could be significantly mitigated. PCR tests completed in 5-10 minutes would greatly shorten the time for information to be received by a patient who may be asymptomatic and shedding virus. PCR tests are far less ambiguous than antibody tests but historically have taken far too long to get test results to the patient.

Optical Train:

See FIG. 5.

In a quest for faster reading of PCR fluorescent signals which indicate the increase of the optical intensity of fluorescent markers as the number of dsDNA increases in a PCR test that is positive for the selected molecular target, the optical train in FIG. 5 has brought a surprising result. The goal of the point-of-care PCR cartridge is to reduce the amount of time commonly experienced in conventional PCR in the preparation steps. Many practitioners in remote areas of the earth need PCR systems that are de-skilled, and which are quick enough to provide a single visit by the patient to the attending physician wherein a diagnosis can be given based on diagnostic tests. Tests which require multiple days for multiple hours are not suitable for the objective of a single visit. This problem exists also in large cities where patients often do not return for a second visit to receive test information and a treatment regime based on that testing information. It is also imperative if one desires to interrupt or slow down the spreading of disease in a pandemic, that is desirable for the disease to be detected in asymptomatic patients prior to having symptoms expressed to aid the diagnostician. It is generally agreed among clinicians that PCR can discover disease organisms prior to a full expression of the disease using PCR tests which confirm the presence of an infective agent in an asymptomatic patient. However, PCR is generally not available in a format that is suitable for this purpose.

Most PCR thermocyclers possess relatively insensitive optical systems and go through several cycles of PCR which produce no measurable results. This is generally referred to as “background”. Once the thermocycler has passed through the background. The fluorescent signal is strong enough to pass an arbitrary luminosity which is commonly referred to as a “crossing threshold”. The general equation for PCR amplification follows the binary expansion function of 2ⁿ where n represents the number of several cycles. The luminosity from fluorescent indicators also follows this function. Many PCR workers have never seen luminosity changes within the first 10 cycles because the luminosity is very small compared to the numbers produced from cycles in the latter teens. However, logically, it would follow, working the function backward, that there ought to be a measurable change in the emission during the first 10 cycles commonly described as “background” luminosity even between cycles one and two though very small. To detect this small change entirely different sensors must be used. It becomes nearly impossible to provide this level of sensitivity in a 96-well PCR thermocycler. However, in a point-of-care device, where the count of test wells is very small and the desire to know the outcome of the test quickly is great, with greatly reduced price of supersensitive sensors, these should be used for very rapid testing. Therefore, the optical system of FIG. 5 becomes feasible where only four PCR vials are expected to be read. The optical system of FIG. 5 is repeated four times, one for each PCR vial.

A Brief Discussion of the Components of the Optical Train:

FIG. 5 Optical Light Train.

8. Special PCR vial (8) which has low intrinsic fluorescence, often colored black to improve the signal-to-noise ratio in extremely low light.

17. The cross-section view of the cartridge (17) base.

26. The cross-section view of the heating and cooling block and PCR vial (8).

34. A miniature laser or LED module, to provide a strong narrow beam at a wavelength which is suitable for excitation of the fluorescent dyes used to indicate the presence of dsDNA such as SYBR® green or the fluorescent molecules which are quenched until the quencher is removed by the action of polymerase during PCR.

35. The beam of light (35) used to excite fluorescent materials within a PCR vial (8).

36. A triangular fixture containing two primary surface mirrors (36). One mirror is used to reflect the light beam of exultation light (35). The other primary mirror surface is used to reflect the emission light (37) from the excited fluorescent materials (41) in a PCR vial (8).

37. The beam of light (37) from the fluorescent material (41) in a PCR vial (8).

39. An optical filter (39) that allows the emitted wavelength of light (37) from a fluorescent material (41) in a PCR vial (8) to pass to the sensor (40) but excludes the lower wavelengths of the exultation beam of light (35).

40. A very low-level light sensor (40) such as a solid-state full photomultiplier capable of photon counting.

51. The light port or tunnel (51) allows the excitation wavelength (35) and the emission wavelength (37) to pass through the cartridge.

41. Represents glowing fluorescent material (41) that has been excited by the excitation wavelength of light (35) coming from the laser or LED (34) and is emitting a wavelength of light (37) different from the exultation wavelength.

Typical Data Graphic of the First 10 PCR Cycles Using a Low-Light Log Sensor:

See FIG. 6.

FIG. 6 is a graphic fluorescent of the first 10 PCR thermal cycles. The top ascending line represents data that correlates with a positive test for the PCR target. The second line which remains nearly at the starting level represents a high correlation with conventional PCR negative test.

### LIST OF REFERENCES

The Following Patents:
U.S. Pat. No. 8,169,122 Incorporated by Reference
U.S. Pat. No. 10,378,045
U.S. Pat. No. 10,960,399
U.S. Pat. No. 10,233,479
U.S. Pat. No. 7,608,399
Japanese patent number 6,087,293
Japanese patent number 6,824,306

List of Illustrations:
FIG. 1. Cross-section PCR Cartridge
FIG. 2. Cross Section Plunger Up
FIG. 3. Cross Section Plunger Down
FIG. 4. Sonication System
FIG. 5. Optical System
FIG. 6. First 10 Cycles

The inventors claim the following:

1. A simplified, self-contained, single-piston, single-actuator, single-pass, vertical PCR cartridge system, comprised of a plurality of components that automatically perform PCR steps, and that require significantly less technical training than traditional methods of PCR testing.

2. The cartridge system of claim 1. which automatically performs the preparatory steps of PCR of extraction and purification of DNA and conversion of RNA to DNA from a plurality of cellular components, viral organisms, bacteria in the spore or vegetative stage, blood, or other DNA containing biological sources exclusively through the use of ultrasonic energy and without the use of additional chemistry or elution of DNA from fiberglass filters or other such commonly used methodologies.

3. The cartridge system of claim 1. which releases said DNA into an aquas fluid exclusively using an ultrasonic sound from an ultrasonic transducer bonded to a flat aluminum circular disk which is 6-10 mm in thickness and has a larger diameter than the centrally located ring-shaped ceramic ultrasonic transducer which is bonded to the aluminum disk with a rigid epoxy. The aluminum disk having a centrally located lumen through which the cartridge system barrel passes but snuggly maintains mechanical contact with the barrel of the cartridge system which comprises a mechanical means of conducting ultrasonic energy originating in the ceramic ring transducer into the cartridge system by way of the aluminum disk.

4. The ultrasonic transducer of claim 3. having an aluminum disk that acts as a heat sink for the heat generated from the ceramic ultrasonic transducer.

5. The ultrasonic transducer of claim 3. which is electronically stimulated with a sinusoidal or a square wave at one of its resonant frequencies thereby transforming the electrical energy into acoustical energy to be delivered into the cartridge system of claim 1. via the aluminum disk of claim 3. for the period of a few seconds to several minutes time.

6. The ultrasonic transducer of claim 3. delivering ultrasonic energy for a period of time being sufficient to release DNA or RNA from its host organism.

7. The cartridge system of claim 1. and the ultrasonic transducer of claim 3. which releases DNA or RNA from its host organism utilizing principles of cavitation created by acoustical waves in an aquas fluid. The phenomenon known as cavitation being similar to that which is used in an ultrasonic cleaning water bath. So-named cavitation bubbles are introduced into the aquas fluid by ultrasonic sound waves. Cavitation bubbles arise and then spontaneously self-destruct releasing a tiny jet of water that has been calculated by others to be in the vicinity of 400 miles an hour when created by the collapsing cavitation bubble. The abrasion caused by these water jets erodes biological entities and attacks their cell walls and other inhibitory structures thus releasing the cellular DNA contents or viral contents which contain either DNA or RNA.

8. The cartridge system of claim 1. having an embedded hydrophobic filter that keeps liquid from flowing into the purification column during sonication.

9. The cartridge system of claim 1. and claim 8. when the sonication process is complete, wherein pressure is applied to the cartridge systems piston to move it downward by a linear actuator exerting force or by manual force. Gradually, the pressure increasing until the fluid pressing against the hydrophobic filter membrane that separates the sonication chamber from the purification chamber of the cartridge system until the wetting pressure of the hydrophobic filter membrane is achieved and becomes wetted allowing liquid to flow freely through the previously hydrophobic filter membrane.

10. The cartridge system of claim 1, wherein the fluid laden with DNA or RNA begins to flow freely into the purification column. If it has been anticipated that RNA will be produced and flow with the fluid going into the purification part of the cartridge system column, there will be reverse transcriptase present in the purification column which will transform the RNA into double-stranded DNA

11. The cartridge system of claim 1, wherein the fluid pressure reaches the wetting pressure of the hydrophobic membrane filter (generally about 20 psi). After the filter is wetted the fluid flows freely into the purification column at the rate specified by the manufacturer of the one-step purification medium. Then the extracted and purified DNA, together, with its fluid pass down through the cartridge system manifold into the PCR reaction vials. As the fluid enters the reaction vials it causes the lyophilized PCR chemistry to immediately hydrate and become active within the fluid. When the reaction vials have filled with fluid the air is forced out of them through capillary vents into sealed chambers in the cartridge system.

12. The tiny capillary vents of claim 11. easily pass air but are resistant to the flow of fluid. When all of the air has been expressed through the capillary tubes into the sealed chambers the pressure from the piston increases to the point where small amounts of residual fluid will then flow into the chambers through the capillary tubes. The design of the flow paths of the cartridge system allows for the PCR vials with aquas fluids and the lyophilized PCR materials to be hydrated before any excess fluid is lost through the vents. No fluid or air contaminated with DNA or residual hazardous biologicals can escape the cartridge system chambers.

13. The cartridge system of claim 1. when the PCR reaction vials are filled with purified DNA together with fluid by the pressure of the piston, then thermocycling may begin with the heating and cooling block. First the block is heated and, in turn, cooled. Times and temperature which may vary from one type of PCR test to another.

14. At the end of each PCR cooling cycle, each of the lasers or laser diodes will be turned on momentarily. The light from the laser or laser diodes enters the cartridge system of claim 1. through a light tunnel and strikes a first primary surface mirror which reflects the light downward at an angle between 95 and 97 degrees into the PCR reaction vials. The wavelength of the laser or laser diodes is the excitation wavelength of the fluorescent materials used to detect double-stranded DNA or to specifically excite the fluorescent materials attached to a target piece of DNA.

15. The fluorescent materials of claim 14. while excited by light of one wavelength emits light at another somewhat higher wavelength. The emitted light then exits the reaction vial and is reflected by a second primary surface mirror of the cartridge system of claim 1. at an angle between 95 and 97 degrees and exits the cartridge system of claim 1. via a light tunnel. In order to block the lower wavelength of the excitation light and to pass on the higher wavelength of emitted light, a high-pass optical filter receives the emitted light and allows it to pass to the photo sensor while blocking the excitation wavelength of light.

16. An optical system capable of reading the output of DNA fluorescent materials during the first ten PCR cycles. The production of meaningful data coming from the first 10 PCR cycles has been demonstrated. PCR technicians have long referred to these early cycles of PCR as unusable background noise. By creating the system capable of reading data from the earliest PCR cycles, we have greatly reduced the time for someone who is awaiting the results of a PCR test.
